(19) United States
(12) Patent Application Publication
(10) Pub. No.: US 2003/0200970 A1
(43) Pub. Date: Oct. 30, 2003

(54) Infant breathing assist apparatus

(76) Inventors: Alex Stenzler, Long Beach, CA (US); Stanley Pechar, Pomona, CA (US); Terry Blansfield, Orange, CA (US); Steve Bredehoft, Orange, CA (US)

Correspondence Address: Kit M. Stetina, STETINA BRUNDA GARRED & BRUCKER, Suite 250, 75 Enterprise, Aliso Viejo, CA, 92656, US

(21) Appl. No.: 10/134,257

(22) Filed: Apr. 29, 2002

(51) Int. Cl. A61M 15/08

(52) U.S. Cl. 128/207.18

(57) ABSTRACT

A breathing apparatus with improved ergonomic aspects to more comfortably accommodate patients breathe while preventing their lungs from collapsing. The present breathing apparatus comprises an apparatus body having proximal and distal ends with at least one hollow passageway extending therebetween. A pair of nostril-engaging stems with generally D-shaped base configurations conform to the anatomy of patient's nostrils and a connector assembly having a rotatable mid portion is used at the apparatus body's distal end to allow moderate movement and thereby reduce torque applied to a patient's face.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

The present invention relates generally to breathing apparatus, and more particularly to an improved breathing apparatus which possesses nostril-engaging stems having generally D-shaped base configurations for better matching the interior anatomical features of patient's nostrils, and further possessing a connector assembly with a rotatable mid portion for providing flexibility so as to reduce undesired torque applied to a patient's face during use.

The use of breathing apparatus upon respiratory-impaired patients is well known. Generally, such apparatus assist in patient breathing by allowing proper exchange of inhaled and exhaled gas while providing pressurized gases to patients' lungs so as to prevent lung collapse. In this way, conventional breathing apparatus operate to allow spontaneous breathing while sustaining a continuous positive airway pressure (cpap) applied to the patients' lungs. One example of such conventional breathing apparatus is shown in U.S. Letters Patent No. 5,193,532 entitled “Device For Generating By Means Of Ejection Action A Continuous Positive Airway (CPAP) During Spontaneous Breathing” issued to Moa et al. on Mar. 16, 1993, the disclosure of which is expressly incorporated herein by reference.

The types of breathing apparatus just mentioned have proven to be effective upon patients whose ability to breathe is impaired. However, although the use of such apparatus has proven generally suitable for their intended purpose, they possess inherent ergonomic and/or anatomical design deficiencies which detract from their overall effectiveness and desirability.

For example, one design deficiency characterizing conventional breathing apparatus is their consistent use of circular-shaped tubes for engaging the patients' nostrils. Even though these circular-shaped tubes generally fit within the patients' nostrils, they fail to match the nostrils' interior anatomical features. As such, patients of all ages have found such prior art devices to be uncomfortable, especially when they are subjugated to such tubes for days or weeks at a time. Further, such circular-shaped tubes are often applied to premature babies and/or neonates with respiratory problems, subjecting them to additional discomfort caused by the tubes.

Another exemplary deficiency marking conventional prior art breathing apparatus is their inability to reduce torque when applied upon the patient's face. Specifically, conventional apparatus are generally composed of rigid structural plastic bodies which fail to yield any significant flexibility during use. In this regard, undesired torque is inevitably applied to the patient's face whenever the apparatus nose piece is caused to move thereabout, such as when repositioning and/or rearranging any of the hoses connecting the breathing apparatus to the ventilator. Such torque occurrence can obviously produce great patient discomfort, particularly in neonate treatment where patient weight is extremely small in comparison to the device.

In view of the above-described ergonomic and/or anatomical design shortcomings of the conventional breathing apparatus, there exists a substantial need in the art for a breathing apparatus which provides anatomically correct nostril-engaging tubes so as to mitigate any unnecessary discomfort to the patient. Furthermore, there exists a substantial need in the art for a breathing apparatus that provides controlled flexibility and/or movement at selected connections to eliminate and/or reduce undesired torque applied to the patient's face during use.

### BRIEF SUMMARY OF THE INVENTION

The present invention specifically addresses and alleviates the above-referenced deficiencies associated with the use of prior art breathing apparatus. More particularly, the present invention comprises a breathing apparatus with improved ergonomic design aspects to more comfortably accommodate patient breathing (e.g., particularly premature babies and/or neonates) while preventing their lungs from collapsing. Thus, the breathing apparatus of the present invention satisfies all of the essential functions of conventional apparatus, such as ventilating patients with atmospheric air and pressurized gas. However, as will become more apparent supra, the present breathing apparatus incorporates certain novel features which significantly increase patient comfort level while performing desired ventilation functions.

In accordance with a preferred embodiment of the present invention, there is provided a breathing apparatus adapted to anatomically conform to the patient's nostrils and reduce undesired torque applied to a patient's face during use. The apparatus of the present invention comprises an elongate apparatus body defining proximal and distal ends, wherein at least one hollow passageway is extended therebetween. Preferably, the apparatus body is formed from polymer/plastic material.

Engaged to the proximal end of the apparatus body is a nose-piece member. In the preferred embodiment, the nose-piece member is removably engaged to the apparatus body at its proximal end and retained there via a frictional fit.

On the opposite exposed end of the nose-piece member, there is formed a pair of outwardly protruding nostril-engaging stems which are insertable within the patients' nostrils. Preferably, the nostril-engaging stems are unitarily formed with the nose-piece member. The stems define flow channels which fluidly communicate with the apparatus body's passageway(s) so as to deliver atmospheric air and pressurized gas to the patient.

Preferably, the nostril-engaging stems utilized with the present breathing apparatus each are formed having an external peripheral wall having a lower base portion which defines a generally D-shaped configuration. Each of the stem's peripheral wall form an annular top portion which transitions into a generally D-shaped configuration at its lower base portion. More specifically, the inward peripheral wall portions at the lower base portion is substantially more flattened than the outward peripheral wall portion of the same. In this respect, the nostril-engaging stems defining generally D-shaped base configurations substantially conform to the interior anatomical features (i.e., septum) of the patients' nostrils, and thus are more comfortably accommodated therein. Preferably, the stems are fabricated from a relatively soft polymer material which can moderately deform to precisely conform to patient specific nostril configurations.

In the preferred embodiment of the present invention, there is further provided a connector assembly which is attached to the apparatus body's distal end. The connector assembly fluidly communicates with the passageway(s) such that it serves as a transition conduit between the breathing hose discharging to ambient atmosphere and the apparatus body. In addition, this assembly defines a connecting end that is axially inserted into the passageway(s) through the apparatus body's distal end. When inserted, the assembly's end is then retained within the passageway(s) via frictional fit.

In accordance with a preferred embodiment of the present invention, the connector assembly joins a hose connector and a passageway connector together at their respective ends, thus forming the mid portion of the breathing assist apparatus assembly.

In the preferred embodiment, the hose connector is sized and configured to rotate or swivel in a plurality of angular orientations relative the passageway connector. To achieve this end, a ball-and-socket joint connection is utilized at the ends of the hose connector and passageway connector. Therefore, in operation, the hose connection allows desired movement relative the mid portion of the apparatus to reduce the torque applied to the patient's face through the nose-piece member engaged thereat.

### BRIEF DESCRIPTION OF THE DRAWINGS

These as well as other features of the present invention will become more apparent upon reference to the drawings wherein:

FIG. 1 is a perspective view of a breathing apparatus utilized upon a patient's face and constructed in accordance with a preferred embodiment of the present invention;

FIG. 2 is an exploded perspective view of the breathing apparatus of FIG. 1 illustrating its apparatus body, a nose-piece member and a connector assembly at respective ends thereof;

FIG. 3 is a plan view of the nose-piece member of FIG. 2 illustrating its nostril-engaging stems each having an external peripheral wall having a top portion which is substantially annular in configuration;

FIG. 4 is a cross-sectional view of the breathing apparatus of FIG. 1 and illustrating one of the hollow passageways that extends therethrough;

FIG. 5 is a perspective view of the nose-piece member of FIG. 2 and illustrating its nostril-engaging stems each having an external peripheral wall having a lower base portion which is generally D-shaped in configuration; and

FIG. 6 is a side view of the nose-piece member of FIG. 5 and illustrating generally flattened inward peripheral wall portions forming the D-shaped configurations at the lower base portions of the nostril-engaging stems.

### DETAILED DESCRIPTION OF THE INVENTION

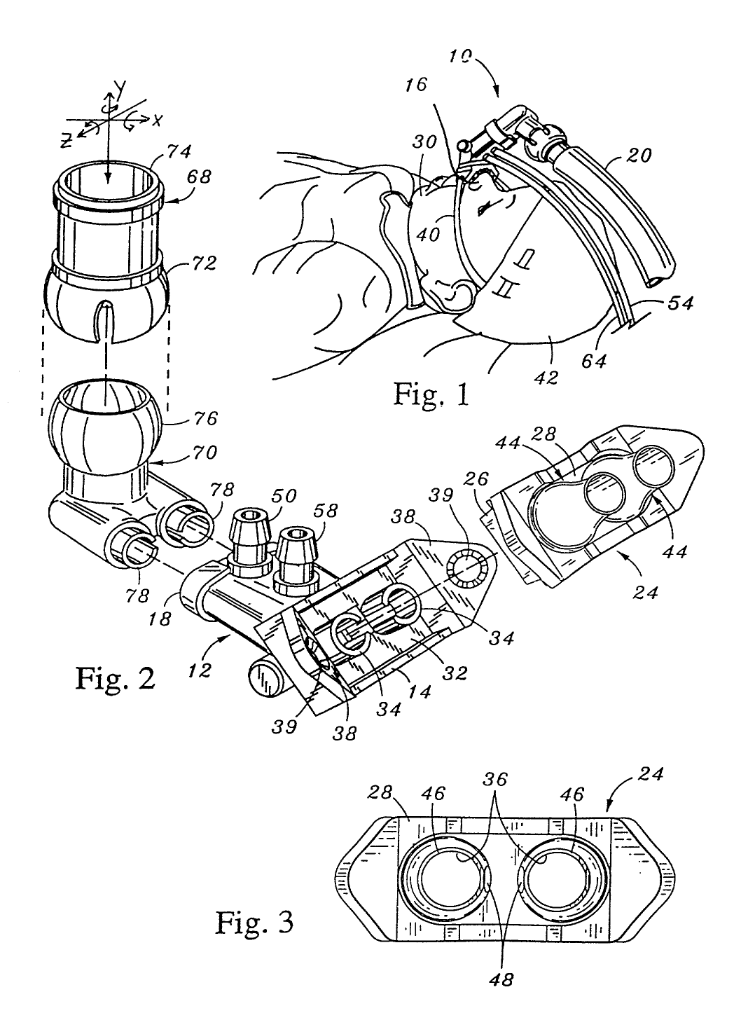

Referring now to the drawings wherein the showings are for purposes of illustrating preferred embodiments of the present invention only, and not for purposes of limiting the same, FIG. 1 perspectively illustrates the breathing apparatus 10 of the present invention. As recited above, the present breathing apparatus 10 possesses improved ergonomic design aspects to more comfortably accommodate patients of all ages and particularly neonates in breathing while preventing their lungs from collapsing. Those of ordinary skill in the art will recognize that the apparatus 10 may be formed to have a variety of configurations, geometries and sizes other than for that shown in the provided figures.

Referring more particularly to FIG. 2, the present breathing apparatus 10 comprises an apparatus body 12. Although the apparatus body 12 is shaped in an elongated manner, such depiction is exemplary in nature and should not be limited thereto. Preferably, the apparatus body is fabricated from a substantially rigid polymer/plastic material.

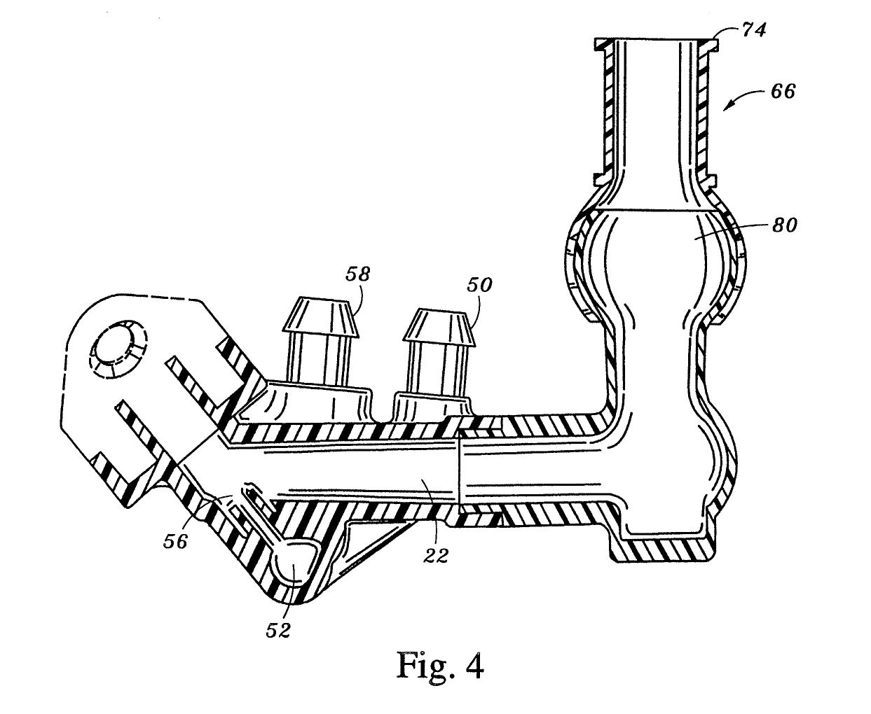

The apparatus body 12 has a proximal end 14 (disposed toward patient's nostrils 16) and a distal end 18 (disposed toward the breathing hose 20). As best shown in FIG. 4, extending between the proximal and distal ends 14, 18 are two passageways 22 which preferably extend in a parallel relationship to each other. However, it will be appreciated by one of ordinary skill in the art that one passageway 22 may be utilized in lieu of two for the intended purpose of conducting inhalable air (i.e., atmospheric air and pressurized gas) and exhaled air therethrough.

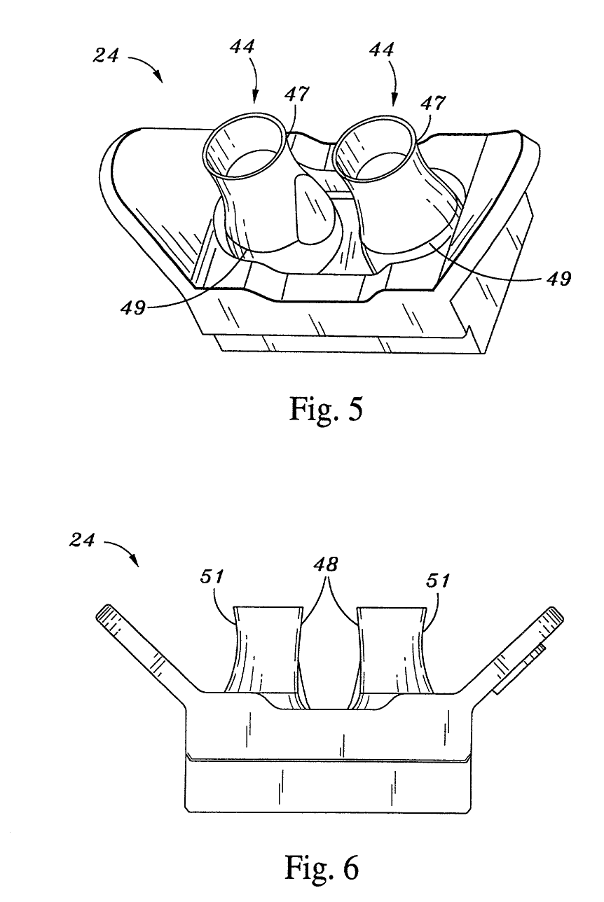

Referring now to FIGS. 2, 3, 5 and 6, a nose-piece member 24 is illustrated having an apparatus end 26 and a patient nose end 28. The nose-piece member 24 may be formed from any suitable material, but is desirably formed from an elastic polymer material, softer than the polymer material of the apparatus body 12 so as to feel soft against the patient skin and moderately deform to the patient's face 30. The nose-piece member 24 includes a pair of outwardly extending nostril engaging stems 44 each having an axially extending aperture 36 extending therethrough. The apparatus end 26 of the nose-piece member 24 is preferably removably mounted to the proximal end 14 of the apparatus body 12. More particularly, the proximal end 14 includes a well 32 having two spaced outwardly extending passageway portals 34. Each of the two passageway portals 34 are in flow communication with a respective one of the passageways 22 extending within the apparatus body 12. Due to the nose-piece member 24 being formed of a preferable elastic polymer material, the apparatus end 26 of the nose-piece member 24 may be removably engaged and retained within the well 32 and upon the passageway portals 34 via frictional fit. As such, the nose apertures/channels 36 fluidly communicate with the passageways 22 via the passageway portals 34.

Preferably, the well 32 has head-engaging portions or flanges 38 which substantially protrude laterally outward therefrom. Each head-portion 38 has a, strap aperture 39 which allows a strap 40 (shown in FIG. 1) to be inserted therethrough so that it can circumscribe the patient's head 42 thereby maintaining the apparatus 10 upon the patient's face 30.

Preferably, the nostril-engaging stems 44 are unitarily formed (i.e., molded) with the nose end 28 of the nose-piece member 24. However, it should be emphasized herein that the nostril-engaging stems 44 could be formed as separate elements.

The nostril-engaging stems 44 each have a peripheral wall 46 with a top portion 47 and a base portion 49. Preferably, the top portion 47 of the peripheral wall 46 is substantially annular in configuration. The annular top portion 47 transitions into a generally D-shaped configuration at its lower base portion 49. More specifically, the inward peripheral wall portion 48 at the base portion 49 is substantially more flattened than the outward peripheral portion 51 of the same. In this respect, these nostril-engaging stems 44 defining generally D-shaped configurations at their respective base portions 49 substantially conform to the interior anatomical features (i.e., septum) of the patient's nostrils 16. Further, due to the nostril-engaging stems being fabricated from an elastic material, the peripheral walls 46 forming the stems 44 may slightly deform upon insertion into the patient's nostrils 16.

Referring now to FIGS. 2 and 4, the apparatus body 12 includes a positive pressure portal 50 for the purpose of sustaining a continuous positive airway pressure to the patient. In this regard, this portal 50 introduces constant air pressure (i.e., atmospheric air and pressurized gas) to the interior of each of the passageways 22. The positive pressure portal 50 is preferably positioned between the two parallel extending passageways 22 so that it can fluidly communicate therewith. The pressure portal 50 may be connected via tubing to a pressurized air/oxygen source (not shown) and is in fluid communication with the passageways 22 via an ejector inlet channel 52 disposed therebetween. The channel 52 forms a manifold splitting fluid flow from the portal 50 into each of the passageways 22. Because the positive pressure portal 50 may be placed in fluid communication with the pressurized air/oxygen source (not shown) by a pressurized gas hose 54, it is able to receive the constant air pressure therefrom and introduce the same into the passageways 22 by using the ejector inlet channel 52.

The ejector inlet channel 52 has a pair of outlet channels 56 which separately extend outwardly to reside within the interior of their respective passageways 22. In addition, each channel 56 is positioned to face toward the proximal end 14, and more particularly to the respective passageway portals 34, so that the channel can effectively direct the constant air pressure thereto. As such, by introducing the constant air pressure into the positive pressure portal 50 and thus into the passageways 22, cpap therapy is provided to reduce the risk of lung collapse.

The apparatus body 12 also includes a pressure sensor portal 58. The pressure sensor portal may lead to an optional conventional pressure sensor (not shown) to measure fluid flow characteristics. As best shown in FIG. 2, as with the pressure gas portal 50, the pressure sensor portal 58 is preferably positioned between the parallel extending passageways 22. The pressure sensor portal 58 is designed in a manner as to fluidly communicate with the passageways 22, and thus with the proximal end 14 of the apparatus body 12.

As illustrated in FIGS. 2 and 4, there is further provided a connector assembly 66 which can be attached to the distal end 18 of the apparatus body 12. The connector assembly 66 defines a substantially hollow interior that allows fluid communication between an ambient atmosphere and the passageways 22. The connector assembly 66 serves as a conduit between the breathing hose 20 (leading to ambient atmosphere) and the apparatus body 12 so that exhaled air from the patient may be properly disposed.

The connector assembly 66 comprises a hose connector 68 and a passageway connector 70 which are movably mounted to one another. More particularly, the hose connector 68 has a hose end 74 and a breathing end 72, whereas the passageway connector 70 has a passageway end 76 and a body end 78. The two connectors 68, 70 can be adjoined to each other by connecting the breathing and passageway ends 72, 76 which assembly comprises the mid portion 80 of the connector assembly 66.

As shown, the mid portion 80 of the connector assembly 66 is sized and configured to rotate in a plurality of angular orientations, that is, rotate about one or more and preferably all of X-Y-Z axes, so as to reduce torque applied to the patient's face 30 during use. In this regard, the rotatability of the mid portion 80 mitigates movement of the nose-piece member 24 when it is being utilized. It is preferred that the hose connector 68 (at its breathing end 72) rotates and/or swivels with respect to the passageway connector 70 (at its passageway end 76).

To achieve this end, a ball-and-socket joint system can be used (as shown in FIG. 2). More particularly, the breathing end 72 may form a ball structure whereas the passageway end 76 may form a complementary socket joint. The ball structure can mate within the complementary socket joint such that it is able to rotate and/or swivel relative to the socket joint (best portrayed in FIG. 4).

The hose end 74 of the hose connector 68 connects to the breathing hose 20 which leads to ambient atmosphere. Moreover, the body end 78 of the passageway connector 70 is axially insertable into the passageways 22 through the apparatus body's distal end 18. Note that FIG. 2 portrays the body end 78 as being two separated ends structurally corresponding to the two passageways 22. When inserted, the body end 78 is then retained within the passageways 22 via frictional fit and also forms coaxial relationships therewith.

In operation, the breathing apparatus 10 of the present invention can be used to more comfortably accommodate patient breathing (e.g., particularly premature babies and/or neonates) while preventing their lungs from collapsing. As illustrated in FIG. 1, the breathing apparatus 10 is applied and maintained upon the patient's face 30 by circumscribing its strap 40 around the patient's head 42. The nostril-engaging stems 44 are inserted within the patient's nostrils 16. Because the peripheral walls 46 of these stems 44 each define an annular top portion 47 which transitions into a generally D-shaped base portion 49, such stems 44 comfortably conform to the interior anatomical features (i.e., septum) of the patient's nostrils 16.

To further increase patient comfort level, the breathing hose connection may be adjusted to reduce the torque applied to the patient's face 30 through the engaged nose-piece member 24. More specifically, the connector assembly's mid portion 80 may be moved in a rotatable/swivel fashion so as to lessen the torque upon the patient's face 30 during use. The rotatability of the mid-portion 80 mitigates movement of the nose-piece member 24 when it is being utilized.

Once the nose-piece member 24 is comfortably accommodated within the patient's nostrils 16, the breathing apparatus 10 is adapted to properly conduct patient breathing. As best shown in FIG. 4, the positive pressure portal 50, which is connected via tubing to a pressurized air/oxygen source, delivers constant air pressure to the patient's nostrils 16 via above-described ejector inlet channels 52. The exhaled air is then disposed to the ambient atmosphere by passing through the passageways 22 which leads to the breathing hose 20. Optionally, the fluid flow characteristics may be measured through the pressure sensor portal 58 which may lead to a conventional pressure sensor.

Additional modifications and improvements of the present invention may also be apparent to those of ordinary skill in the art. Thus, the particular combination of parts described and illustrated herein is intended to represent only certain embodiments of the present invention, and is not intended to serve as limitations of alternative devices within the spirit and scope of the invention.

What is claimed is:

1. An apparatus to assist in breathing while anatomically conforming to patient's nostrils, the apparatus comprising:
an elongated apparatus body having proximal and distal ends and defining at least one hollow passageway extending therebetween;
nostril-engaging stems sized and configured to outwardly protrude from the proximal end and fluidly communicating with the passageway(s), each of the nostril-engaging stems having a peripheral wall with top and base portions, each of the base portions forming a D-shaped configuration to anatomically conform to the patient's nostrils; and
wherein atmospheric air and pressurized gas flow from the passageway(s) to the proximal end so as to be supplied to the patient's nostrils when the nostril-engaging stems are engaged therein.

2. The apparatus of claim 1 wherein the apparatus body is fabricated from a plastic material.

3. The apparatus of claim 1 wherein the at least one passageway comprises two passageways extending in a parallel relationship to each other between the proximal and distal ends of the apparatus body.

4. The apparatus of claim 1 wherein the apparatus body comprises a positive pressure portal fluidly communicating with the passageway(s) to introduce the atmospheric air and pressurized gas thereinto.

5. The apparatus of claim 4 wherein the positive pressure portal is in fluid communication with the passageway(s) via an ejector inlet channel.

6. The apparatus of claim 5 wherein the ejector inlet channel has at least one inlet end disposed within the passageway(s) and facing toward the proximal end for guiding the atmospheric air and pressurized gas thereto.

7. The apparatus of claim 1 wherein the apparatus body comprises a pressure sensor portal fluidly communicating with the proximal end.

8. The apparatus of claim 1 wherein the nostril-engaging stems are fabricated from an elastic material.

9. The apparatus of claim 1 wherein each of the top portions have a substantially annular configuration.

10. The apparatus of claim 1 further comprising a nose-piece member having apparatus and nose ends, the apparatus end being engaged with the proximal end, the nose end forming the outwardly protruding nostril-engaging stems thereon.

11. The apparatus of claim 10 wherein the apparatus end is removably engaged with the proximal end of the apparatus body.

12. The apparatus of claim 10 wherein the apparatus end is engaged with the proximal end of the apparatus body via a frictional fit.

13. The apparatus of claim 10 wherein the nostril engaging stems are unitarily formed on the nose end of the nose-piece member.

14. The apparatus of claim 10 wherein the nose-piece member defines nose channels to fluidly communicate the passageway(s) with the nose-engaging stems.

15. An apparatus to assist in breathing while reducing torque applied to a patient's face, the apparatus comprising:
an elongated apparatus body having proximal and distal ends and defining at least one hollow passageway extending therebetween; and
a connector assembly engaged to the distal end and fluidly communicating with the passageway(s), the connector assembly having a mid portion sized and configured to allow relative rotation between the apparatus body and the connector assembly to reduce torque applied to the patient's face when the proximal end is fitted thereon.

16. The apparatus of claim 15 wherein the apparatus body is fabricated from a plastic material.

17. The apparatus of claim 15 wherein the at least one passageway comprises two passageways extending in a parallel relationship to each other between the proximal and distal ends of the apparatus body.

18. The apparatus of claim 15 wherein the apparatus body comprises a positive pressure portal fluidly communicating with the passageway(s) to introduce atmospheric air and pressurized gas thereinto.

19. The apparatus of claim 15 wherein the apparatus body comprises a pressure sensor portal fluidly communicating with the proximal end.

20. The apparatus of claim 15 wherein the connector assembly comprises a hose connector with a breathing end and a passageway connector with a passageway end, the breathing and passageway ends jointly forming the rotatable mid portion when connected together.

21. The apparatus of claim 20 wherein the breathing and passageway ends are sized and configured to swivel with respect to each other.

22. The apparatus of claim 21 wherein a ball-and-socket joint connection is formed between the breathing and passageway ends.

23. The apparatus of claim 20 wherein the passageway connector comprises a body end for engaging the distal end of the apparatus body.

24. The apparatus of claim 23 wherein the body end is axially insertable into the passageway(s) through the distal end of the apparatus body.

25. The apparatus of claim 24 wherein the body end is retained within the passageway(s) via a frictional fit and forming a coaxial relationship therewith.

26. The apparatus of claim 15 further comprising a nose-piece member having apparatus and nose ends, the apparatus end being engaged with the proximal end of the apparatus body, the nose end forming outwardly protruding nostril-engaging stems adapted to be fitted to the patient's face.
